(19) United States
(12) Patent Application Publication
(10) Pub. No.: US 2011/0238133 A1
(43) Pub. Date: Sep. 29, 2011

(54) ELECTRICAL STIMULATION OF THE EYE

(75) Inventor: Yossi Gross, Moshav Mazor (IL)

(73) Assignee: RAINBOW MEDICAL LTD., Herzliya (IL)

(21) Appl. No.: 13/033,742

(22) Filed: Feb. 24, 2011

Related U.S. Application Data
(63) Continuation-in-part of application No. 11/995,904, filed on Mar. 11, 2008.
(60) Provisional application No. 60/702,491, filed on Jul. 25, 2005.
(60) Provisional application No. 60/721,728, filed on Sep. 28, 2005.

(51) Int. Cl. A61F 9/00

(52) U.S. Cl. 607/53

(57) ABSTRACT

Apparatus and methods are described for treating an eye of a subject. One or more electrodes are placed at least partially inside the subject's eye. A control unit treats the eye by driving the electrodes to apply an electrical signal to the eye that induces production of nitric oxide (NO). Other embodiments are also described.

### CROSS REFERENCES TO RELATED APPLICATIONS

The present application is a continuation in part of U.S. patent application Ser. No. 11/995,904 to Gross, filed Mar. 11, 2008, which is the U.S. national phase of PCT Application No. PCT/IL06/000856 to Gross, filed Jul. 25, 2006, entitled “Electrical stimulation of blood vessels,” which claims the benefit of: (a) U.S. Provisional Application 60/702,491, filed Jul. 25, 2005, entitled, “Electrical stimulation of blood vessels,” and (b) U.S. Provisional Application 60/721,728, filed Sep. 28, 2005, entitled, “Electrical stimulation of blood vessels.” All of the aforementioned applications are incorporated herein by reference.

### FIELD OF EMBODIMENTS OF THE INVENTION

Some applications of the present invention generally relate to medical apparatus. Specifically, some applications of the present invention relate to electrical stimulation of the eye.

### BACKGROUND

Glaucoma is a disease which can lead to irreversible loss of vision. A significant risk factor for glaucoma is ocular hypertension, i.e., increased pressure within the eye. Schlemm's canal is a circular channel in the eye that collects aqueous humor from the anterior chamber and delivers it into the bloodstream. On the inside of the canal, nearest to the aqueous humor, it is covered by the trabecular meshwork. If debris builds up, due to infection or injury in the aqueous humor, the canal typically becomes blocked, thereby causing glaucoma.

Canaloplasty is a procedure in which an incision is made into the eye to gain access to Schlemm's canal. A microcatheter circumnavigates the canal around the iris, enlarging the main drainage channel and smaller collector channels through the injection of a sterile, gel-like material called viscoelastic. The catheter is then removed, and a suture is placed within the canal and tightened. By opening the canal, the pressure inside the eye may be relieved.

### SUMMARY OF EMBODIMENTS

For some applications of the present invention, one or more electrodes are placed in or adjacent to an eye of a subject, and are driven to apply a signal that induces NO production adjacent to the electrodes, in order to treat an eye disease. For example, the increased NO production may be used to reduce intraocular pressure by dilating the Schlemm's canal, in order to treat glaucoma. Alternatively or additionally, increased blood flow induced by arterial dilation caused by the NO production minimizes optic nerve degeneration, such as is seen in age-related macular degeneration (AMD). Alternatively or additionally, the increased blood flow in one or more retinal arteries treats or prevents diabetic retinopathy.

There is therefore provided, in accordance with some applications of the present invention, apparatus for treating an eye of a subject, the apparatus including:

one or more electrodes, configured to be placed at least partially inside the subject's eye; and

a control unit, configured to treat the eye by driving the electrodes to apply an electrical signal to the eye that induces production of nitric oxide (NO).

For some applications, the control unit is configured to drive the electrodes to apply the signal to induce the NO production sufficiently to induce dilation of a retinal artery of the subject.

For some applications, the control unit is configured to drive the electrodes to apply the signal to induce the NO production sufficiently to minimize degeneration of an optic nerve of the subject.

For some applications, at least one of the electrodes includes a coil that is configured to receive energy inductively from the control unit.

For some applications, the control unit is configured to be placed at least partially in the eye, and the apparatus includes a charging coil that is configured to power the control unit.

For some applications, the charging coil is configured to be coupled to glasses of the subject.

For some applications, the apparatus further includes a photovoltaic cell, and the charging coil is configured to power the control unit by extracting power from the photovoltaic cell.

For some applications, the apparatus further includes a receiving coil configured to be placed inside the subject's eye and to receive power from the charging coil.

For some applications, the receiving coil is configured to be placed inside a Schlemm's canal of the subject's eye.

For some applications, the charging coil is configured to clean a trabecular meshwork of the subject's eye by vibrating the receiving coil, by generating a magnetic field.

For some applications, the receiving coil is configured to mechanically dilate the Schlemm's canal.

For some applications, the receiving coil is configured to loop around substantially all of the Schlemm's canal.

For some applications, the receiving coil forms a closed loop that is configured to loop around a portion of the Schlemm's canal.

For some applications, the receiving coil includes a plurality of receiving loops configured to be oriented such that a plane defined by each of the receiving loops is substantially perpendicular to a local longitudinal axis of the Schlemm's canal.

For some applications, at least one of the electrodes is configured to be placed at a site posterior to a retina of the eye.

For some applications, the control unit is configured to drive the electrodes to apply the signal to induce the NO production sufficiently to reduce an intraocular pressure of the eye.

For some applications, the electrodes are configured to be placed around a cornea of the eye, and the control unit is configured to drive the electrodes to apply the signal to induce the NO production in a vicinity of a trabecular meshwork of the eye.

For some applications, the electrodes are configured to be placed in a Schlemm's canal of the subject's eye.

For some applications, the electrodes are configured to mechanically dilate the Schlemm's canal.

There is further provided, in accordance with some applications of the present invention, a method for treating a subject, including:

identifying that the subject suffers from a condition of an eye; and

treating the eye condition by inducing production of nitric oxide (NO) in the eye by applying an electrical signal to the eye.

The present invention will be more fully understood from the following detailed description of embodiments thereof, taken together with the drawings, in which:

### BRIEF DESCRIPTION OF THE DRAWINGS

FIG. 1 is a schematic illustration of an electrode device implanted in a subject's eye, and a charging coil disposed on the subject's glasses, in accordance with some applications of the present invention;

FIGS. 2A-B are schematic illustrations of the electrode device implanted around the subject's cornea, in accordance with some applications of the present invention; and

FIGS. 3A-C are schematic illustrations of electrode devices, in accordance with some applications of the present invention.

### DETAILED DESCRIPTION OF EMBODIMENTS

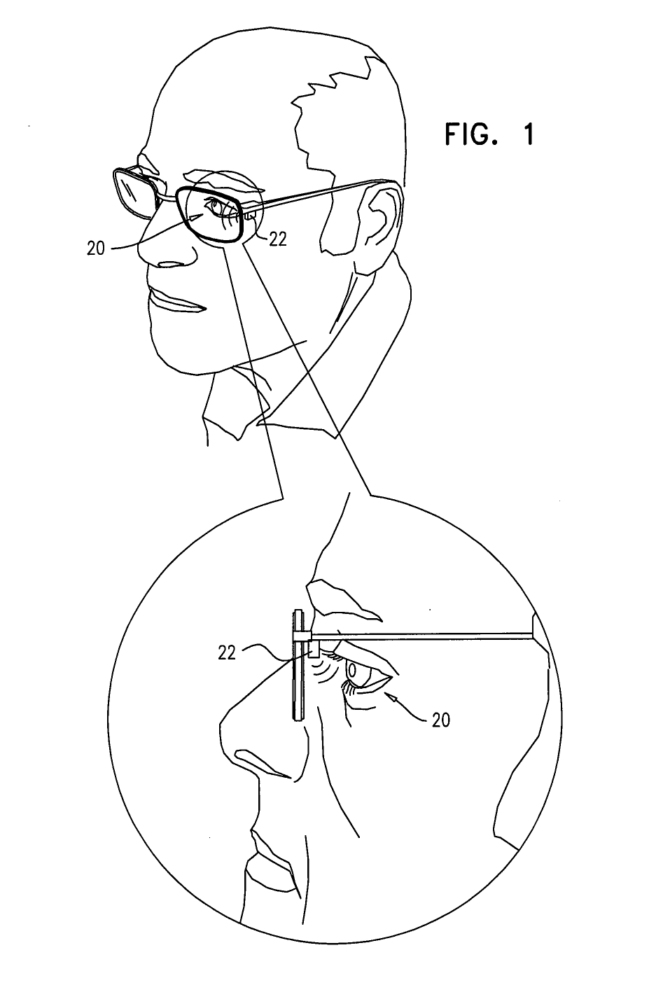

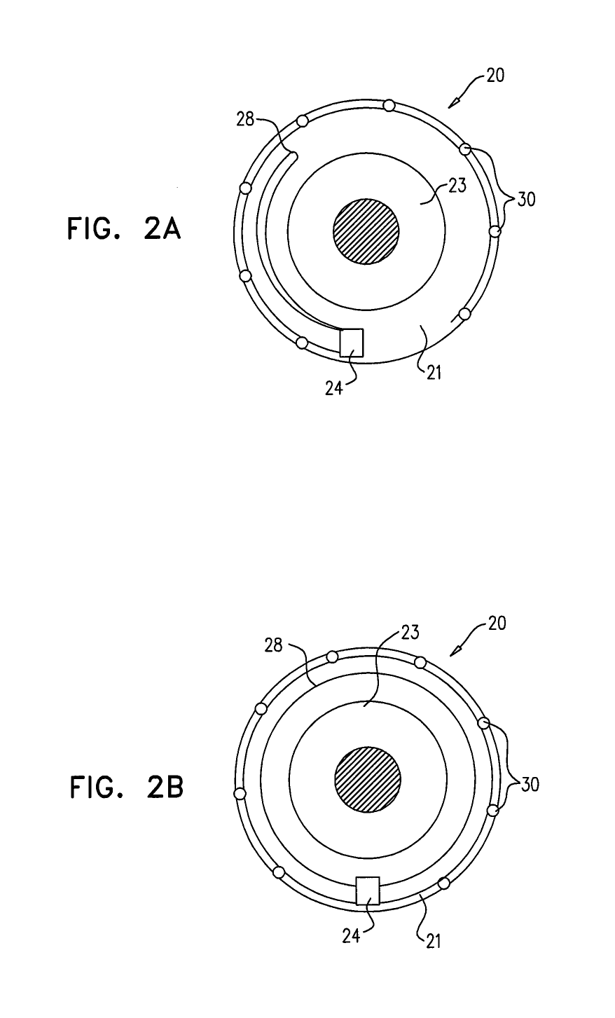

Reference is now made to FIG. 1, which is a schematic illustration of an electrode device 20 implanted in a subject's eye, and a charging coil 22 disposed on the subject's glasses, in accordance with some applications of the present invention. For some applications, one or more electrodes 30 (shown in FIGS. 2A-B and 3A-C) of the electrode device are placed in or adjacent to the subject's eye, and are driven to apply a signal that induces NO production adjacent to the electrodes, in order to treat an eye disease. For some applications, the electrodes are powered by charging coil 22 that is disposed outside the subject's eye, e.g., coupled to the subject's glasses, as shown.

For example, the increased NO production may be used to reduce intraocular pressure, in order to treat glaucoma, as described in further detail hereinbelow. Alternatively or additionally, increased blood flow induced by the NO production minimizes optic nerve degeneration, such as is seen in age-related macular degeneration (AMD). Alternatively or additionally, as noted hereinabove, the increased blood flow in one or more retinal arteries treats or prevents diabetic retinopathy.

Reference is now made to FIGS. 2A-B, which are schematic illustrations of electrode device 20 implanted in the subject's Schlemm's canal 21, around the subject's cornea 23, in accordance with some applications of the present invention. (It is noted that portions of the subject's anatomy, particularly the cornea and the Schlemm's canal, are not drawn to scale in FIGS. 2A-B, in order to emphasize certain features of the anatomy.)

For some applications, electrode device 20 includes a control unit 24, a receiving coil 28 and a plurality of electrodes 30. The receiving coil receives power transmitted into the eye from outside the eye by charging coil 22 (shown in FIG. 1). Electrodes 30 (e.g., exactly two electrodes, or more than two electrodes, as shown) are placed around cornea 23 inside Schlemm's canal 21. Control unit 24 drives the electrodes to apply a signal that induces NO production in the Schlemm's canal, i.e., in a vicinity of the trabecular meshwork of the eye. Such NO production enhances the flow of aqueous humor through the trabecular meshwork to Schlemm's canal 21, thereby reducing intraocular pressure to treat glaucoma, for example, by dilating the Schlemm's canal.

For some applications, receiving coil 28 makes a closed loop inside a portion of the Schlemm's canal that surrounds a portion of the cornea (e.g., less than 50% of the circumference of the cornea, or more than 50% of the circumference of the cornea), as shown in FIG. 2A. Alternatively, the receiving coil loops around the whole cornea, as shown in FIG. 2B. For some applications control unit 24 is not powered by a charging coil that is disposed outside the subject's eye. For example, control unit 24 may include a photovoltaic cell, or a separate photovoltaic cell may be disposed inside the subject's eye, the photovoltaic cell being configured to power the control unit. Alternatively or additionally, control unit 24 may be powered by a battery. For some applications, control unit 24 is not disposed inside the subject's eye.

For some applications, charging coil 22 generates a magnetic field that drives electrode device 20 to vibrate inside the Schlemm's canal. For example, drainage of aqueous humor fluid via the trabecular meshwork may be enhanced, by cleaning the trabecular meshwork, by vibrating the electrode device.

For some applications of the present invention, electrode device 20 is adapted to be placed in an eye of a subject, and to induce dilation of a retinal artery, for example, to treat diabetic retinopathy. For some applications, electrode device 20 comprises a coil (e.g., coil 28), which receives energy inductively in order to drive current into the tissue in contact therewith or adjacent thereto. The energy may be released by inductively driving current flow in the tissue, or via an electrode coupled to the coil. As appropriate based on surgical constraints or the particular pathology to be treated, the coil may be placed in an anterior or posterior position within the globe of the eye, or, alternatively, posterior to the retina.

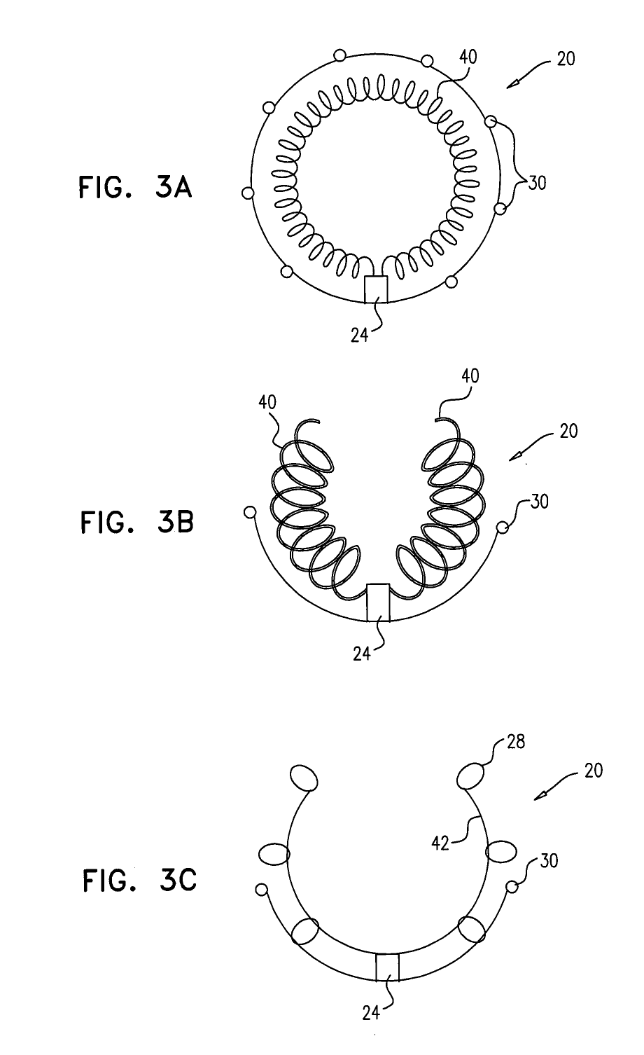

Reference is now made to FIGS. 3A-B, which are schematic illustrations of electrode device 20, in accordance with some applications of the present invention. For some applications, the electrode device includes a coiled loop 40. The coiled loop 40 acts to mechanically dilate the Schlemm's canal. Thus, the electrode device mechanically dilates the Schlemm's canal, as well as inducing dilation of the Schlemm's canal via NO production. For some applications coiled loop 40 acts as (a) electrodes 30, control unit 24 driving a current into the Schlemm's canal via the coiled loop (application not shown), and/or (b) receiving coil 28, control unit 24 receiving power from charging coil 22 (shown in FIG. 1), via the coiled loop (application shown in FIGS. 3A-B). For some applications, the coiled loop loops around the whole cornea, as shown in FIG. 3A. Alternatively a single coiled loop or a plurality of coiled loops make one or more closed loops inside one or more portions of the Schlemm's canal that surround one or more portions of the cornea, as shown in FIG. 3B.

Reference is now made to FIG. 3C, which is a schematic illustration of electrode device 20, in accordance with some applications of the present invention. For some applications, receiving coil 28 includes a plurality of receiving loops that are disposed on a support structure 42. The receiving loops are oriented such that a plane defined by each of the receiving loops is generally perpendicular to the local longitudinal axis of the Schlemm's canal. The receiving loops act to mechanically dilate the Schlemm's canal. Thus, the electrode device mechanically dilates the Schlemm's canal, as well as inducing dilation of the Schlemm's canal via NO production. For some applications, support structure 42 loops around the whole cornea (application not shown). Alternatively, the support structure loops around a portion of the Schlemm's canal that surrounds a portion of the cornea, as shown in FIG. 3C. For some applications, the loops that are disposed on support structure 42 act as electrodes 30, control unit 24 driving a current into the Schlemm's canal via the loops (application not shown).

For some applications, apparatus and methods described herein are combined with apparatus and methods described in U.S. patent application Ser. No. 11/995,904 to Gross (published as US 2008/0215117 to Gross), which is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating an eye of a subject, the apparatus comprising:
one or more electrodes, configured to be placed at least partially inside the subject's eye; and
a control unit, configured to treat the eye by driving the electrodes to apply an electrical signal to the eye that induces production of nitric oxide (NO).

2. The apparatus according to claim 1, wherein the control unit is configured to drive the electrodes to apply the signal to induce the NO production sufficiently to induce dilation of a retinal artery of the subject.

3. The apparatus according to claim 1, wherein the control unit is configured to drive the electrodes to apply the signal to induce the NO production sufficiently to minimize degeneration of an optic nerve of the subject.

4. The apparatus according to claim 1, wherein at least one of the electrodes comprises a coil that is configured to receive energy inductively from the control unit.

5. The apparatus according to claim 1, wherein the control unit is configured to be placed at least partially in the eye, wherein the apparatus comprises a charging coil that is configured to power the control unit.

6. The apparatus according to claim 5, wherein the charging coil is configured to be coupled to glasses of the subject.

7. The apparatus according to claim 5, further comprising a photovoltaic cell, wherein the charging coil is configured to power the control unit by extracting power from the photovoltaic cell.

8. The apparatus according to claim 5, further comprising a receiving coil configured to be placed inside the subject's eye and to receive power from the charging coil.

9. The apparatus according to claim 8, wherein the receiving coil is configured to be placed inside a Schlemm's canal of the subject's eye.

10. The apparatus according to claim 9, wherein the charging coil is configured to clean a trabecular meshwork of the subject's eye by vibrating the receiving coil, by generating a magnetic field.

11. The apparatus according to claim 9, wherein the receiving coil is configured to mechanically dilate the Schlemm's canal.

12. The apparatus according to claim 9, wherein the receiving coil is configured to loop around substantially all of the Schlemm's canal.

13. The apparatus according to claim 9, wherein the receiving coil forms a closed loop that is configured to loop around a portion of the Schlemm's canal.

14. The apparatus according to claim 9, wherein the receiving coil comprises a plurality of receiving loops configured to be oriented such that a plane defined by each of the receiving loops is substantially perpendicular to a local longitudinal axis of the Schlemm's canal.

15. The apparatus according to claim 1, wherein at least one of the electrodes is configured to be placed at a site posterior to a retina of the eye.

16. The apparatus according to claim 15, wherein the control unit is configured to drive the electrodes to apply the signal to induce the NO production sufficiently to reduce an intraocular pressure of the eye.

17. The apparatus according to claim 16, wherein the electrodes are configured to be placed around a cornea of the eye, and wherein the control unit is configured to drive the electrodes to apply the signal to induce the NO production in a vicinity of a trabecular meshwork of the eye.

18. The apparatus according to claim 17, wherein the electrodes are configured to be placed in a Schlemm's canal of the subject's eye.

19. The apparatus according to claim 18, wherein the electrodes are configured to mechanically dilate the Schlemm's canal.

20. A method for treating a subject, comprising:
identifying that the subject suffers from a condition of an eye; and
treating the eye condition by inducing production of nitric oxide (NO) in the eye by applying an electrical signal to the eye.

21. The method according to claim 20, wherein applying the signal comprises configuring the signal to induce the NO production sufficiently to induce dilation of a retinal artery of the subject.

22. The method according to claim 20, wherein applying the signal comprises configuring the signal to induce the NO production sufficiently to minimize degeneration of an optic nerve of the subject.

23. The method according to claim 20, wherein applying the electrical signal comprises placing a photovoltaic cell in the eye, and powering the application of the signal using power generated by the cell.

24. The method according to claim 20, wherein applying the signal comprises receiving energy inductively within the eye, and applying the signal using the received energy.

25. The method according to claim 20, wherein applying the signal comprises applying the signal at a site posterior to a retina of the eye.

26. The method according to claim 20, wherein applying the signal to the eye comprises placing electrodes inside the eye and powering the electrodes from outside the eye.

27. The method according to claim 26, wherein powering the electrodes comprises powering the electrodes via a charging coil that is coupled to glasses of the subject.

28. The method according to claim 26, wherein powering the electrodes comprises extracting power from a photovoltaic cell that is electrically coupled to the charging coil.

29. The method according to claim 26, wherein powering the electrodes comprises receiving power from the charging coil using a receiving coil that is placed inside the subject's eye.

30. The method according to claim 29, wherein receiving the power comprises receiving the power using a receiving coil that is placed inside a Schlemm's canal of the subject's eye.

31. The apparatus according to claim 30, the method further comprising cleaning a trabecular meshwork of the subject's eye by vibrating the receiving coil, by generating a magnetic field.

32. The method according to claim 30, further comprising mechanically dilating the Schlemm's canal using the receiving coil.

33. The method according to claim 30, wherein receiving the power comprises receiving the power using a receiving coil that loops around substantially all of the Schlemm's canal.

34. The method according to claim 30, wherein receiving the power comprises receiving the power using a receiving coil that forms a closed loop that loops around a portion of the Schlemm's canal.

35. The method according to claim 30, wherein receiving the power comprises receiving the power using a plurality of receiving loops that are oriented such that a plane defined by each of the receiving loops is substantially perpendicular to a local longitudinal axis of the Schlemm's canal.

36. The method according to claim 20, wherein the identifying comprises identifying that the subject suffers from glaucoma, and wherein applying the signal comprises configuring the signal to induce the NO production sufficiently to reduce an intraocular pressure of the eye.

37. The method according to claim 36, wherein applying the signal comprises applying the signal around a cornea of the eye to induce the NO production in a vicinity of a trabecular meshwork of the eye.

38. The method according to claim 37, wherein applying the signal comprises applying the signal to a Schlemm's canal of the subject's eye.

39. The method according to claim 38, further comprising mechanically dilating the Schlemm's canal.
